(19) United States
(12) Patent Application Publication
(10) Pub. No.: US 2019/0128876 A1
(43) Pub. Date: May 2, 2019

(54) Rapid multi-path micro-lens imaging ultra-micro immunoassay apparatus

(71) Applicant: JINAN UNIVERSITY, Guangdong (CN)

(72) Inventors: Yaoxiong HUANG, Guangdong (CN); Jiang He, Guangdong (CN)

(21) Appl. No.: 16/220,040

(22) Filed: Dec. 14, 2018

Related U.S. Application Data
(63) Continuation-in-part of application No. PCT/CN2017/076564, filed on Mar. 14, 2017.

(30) Foreign Application Priority Data
Oct. 10, 2016 (CN) 201610885630.0

(51) Int. Cl. G01N 33/53; G01N 21/64; G01N 21/76

(52) U.S. Cl. CPC G01N 33/5302; G01N 21/6486; G01J 1/0403; G01N 21/6452; G01N 21/6456; G01N 21/76

(57) ABSTRACT

The presently claimed invention applies to an immunoassay apparatus based on micro-lens imaging technique and related product. In a preferred embodiment, the immunoassay apparatus comprises a monochromatic light source module, an intelligent auto-scanning and imaging module, a temperature controlled transparent toughened glass platform, a central control module, and a touchscreen displaying module. By automatically performing scan in focus imaging on the micro-lenses immersing in antigen-antibody solution and analyzing the data of the image, the apparatus can monitor the refractive index change of a sample solution before and after antigen-antibody reaction, so as to determine the concentration of antigen or antibody in the solution without requiring any labeling, expensive enzymes, pre-immobilization/modification, and post-washing. It can detect ultra-micro amount of Ag/Ab (˜pg/mL) in very low sample volume solution (several μL) within 2 minutes. It is of high accuracy and reliability. Moreover, the apparatus is simple and compact, can be portable for on-site immunoassay.

### CROSS REFERENCE TO RELATED APPLICATIONS

This application claims priority to China Application Number PCT/CN2017/076564 filed on Mar. 14, 2017.

### BACKGROUND OF INVENTION

### Technical Field

The claimed invention is related to optical technology, particularly involving an immunoassay apparatus by which chemical and biochemical assays such as antigen and antibody detection are performed by monitoring the variation of refractive index in sample solution with a technique of micro-lens imaging.

### Description of Related Art

Known methods exist for antigen (Ag) and antibody (Ab) detection, but the current methods have inherent limitations. Such as the method of ELISA, it is not only complicated to operate and very time consuming for detection, but also requires pre-immobilization or modification, and post-washing, and needs expensive enzymes as amplifier.

Another major method currently in use is the Surface Plasmon Resonance (SPR). Besides requiring pre-immobilization, SPR needs very expensive instrument for detection. Other conventional methods such as Fluorescence and Chemiluminescence Immunoassays, also need labeling and expensive reagents and equipment. The detection performed by the methods is subjective with cross reactivity.

These techniques cannot simultaneously satisfy the current requirements of Ag-Ab detection: objective detection with high accuracy, reliability and sensitivity (detection limit: ˜pg−ng/mL); without requiring any labeling, expensive enzyme, pre-immobilization or modification, and post-washing; of capability to perform detection on a small volume of samples (˜μL) in a simple and easy manner within a short time (˜2 minutes); with a simple device portable for on-site analysis. As a consequence, alternate approaches to immunoassay apparatus are desirable.

### BRIEF SUMMARY OF THE INVENTION

According to the presently claimed invention, its object is to provide a rapid multi-path high sensitivity micro-lens imaging ultra-micro immunoassay apparatus which can overcome the limitations of conventional immunoassay apparatuses and simultaneously satisfy all the current requirements of Ag-Ab detection mentioned in

In particular, by using micro-lens imaging technique, it can detect Ag or Ab presenting in sample solution in both qualitative and quantitative manner, while without using any labeling, expensive enzymes, pre-immobilization/modification, and post-washing. The detection is objective, can be performed on very low sample volume (several μL) and finished in 2 minutes. It is of high accuracy, reliability, and its detection limit is as low as pg/mL. Moreover, the apparatus is simple and low cost, can be portable for on-site immunoassay.

In order to achieve the above mentioned object, the immunoassay apparatus according to the presently claimed invention is a compact apparatus which comprises: a monochromatic light source module, an intelligent auto-scanning and imaging module, a temperature controlled transparent toughened glass platform, a central control module with specially designed software unit for intelligent micro-lens image auto-recognition and data analysis, and a touchscreen displaying module.

According to another aspect of the invention, said monochromatic light source module emits monochromatic parallel light projecting to the spherical top of micro-lens with an angle of divergence less than 0.5° and a beam size greater than the size of micro-lens, to provide illumination for micro-lens imaging.

According to still another aspect of the invention, said intelligent auto-scanning and imaging module comprises a galvanometer optical scanner, a high resolution digital camera with pixels more than 10 M and frame rate ≥10 fps, an autofocus objective lens, a driving motor and a driving control circuit board to control said optical scanner and said driving motor for auto-scanning and autofocus imaging of micro-lens. This enables to rapidly and automatically obtain in focus image of micro-lenses.

According to still another aspect of the invention, said temperature controlled transparent toughened glass platform comprises a transparent toughened glass board with a thin film of IndiumTinOxide and a high accuracy PID temperature controller, so that micro-lens multi-well plate can be placed on it with good thermal contact for temperature control and excellent transmittance for high-quality imaging of said micro-lens, the temperature of the sample solution in said micro-lens multi-wells plate can reach to set temperature within 2 min and with accuracy of ±0.1° C.

According to still another aspect of the invention, said central control module includes a microcomputer with specially designed software unit for controlling the operations of said apparatus including auto-scanning, autofocus imaging, intelligent image auto-recognition and data analysis, said microcomputer is of a size (15 cm×15 cm×5 cm), CPU 2 G Hz, memory ≥2G, hard disk ≥100 G, this enables said central control module to be integrated into a main box of said portable compact immunoassay apparatus and operate smoothly.

According to still another aspect of the invention, said intelligent image auto-recognition and data analysis software unit is able to perform rapid intelligent image auto-recognition and data analysis on the image of said micro-lens and monitor the instantaneous variation of the refractive index during the antigen-antibody reaction process in sample solution, this enables said apparatus to determine the concentration of antigen/antibody in sample solution by comparing its refractive indices before and after antigen-antibody reaction.

According to still another aspect of the invention, said touchscreen has data displaying windows and control touch keys for the operations of imaging, image recognition, data analysis and displaying.

### BRIEF DESCRIPTION OF THE DRAWINGS

For a full understanding of the nature and objects of the present invention, reference should be had to the following detailed description taken in connection with the accompanying drawings in which like numerals in the several drawings are employed to denote like parts, and wherein:

FIG. 1 shows the structure of said compact immunoassay apparatus 10.

FIG. 2 is the structure of said intelligent auto-scanning and imaging module 104.

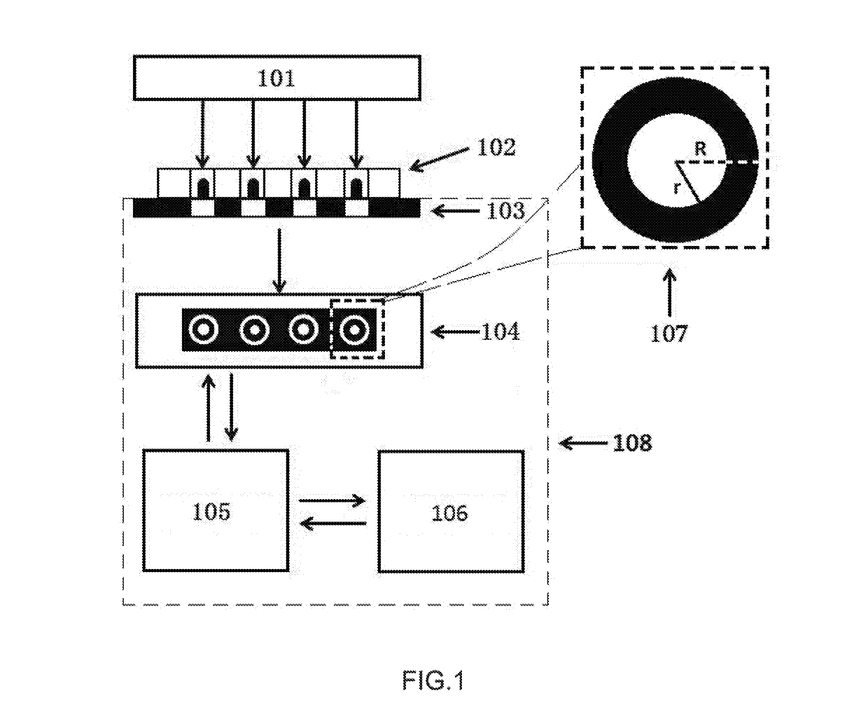

Referring to FIG. 1, there is shown exemplary apparatus 10 for assaying a sample solution, which apparatus incorporates the principles of the present invention. In a preferred embodiment, when a small drop of sample solution is dropped into the sample wells of said micro-lens multi-well test plate 102 which is placed on said temperature controlled transparent toughened glass platform 103, by the illumination of said monochromatic light source module 101, said intelligent auto-scanning and imaging module 104 automatically performs optical scanning and focusing on the micro-lenses immersed in the sample solution in said micro-lens multi-well plate 102 to obtain distinct image 107 of said micro-lenses. Then said central control module 105 works with said intelligent image auto-recognition and data analysis software unit to intelligently recognize the image of micro-lens 107 displaying in said touchscreen displaying module 106, which is a round image with an outer edge that is a black ring, and obtain the values of r and R in the black ring from the image of micro-lens 107.

With the values of r and R, said central control module 105 works with said intelligent image auto-recognition and data analysis software unit to deduce the refractive index n₁ of the sample solution, by having the refractive index n₂ of the micro-lens and using the following equation:

$$\frac{r}{R} = sin α - ( cos α + \frac{h - R}{R} ) \frac{sin α \sqrt{1 - {k}^{2} {sin}^{2} α} - k sin α cos α}{cos α \sqrt{a - {k}^{2} {sin}^{2} α} + k {sin}^{2} α} , \qquad ( 1 )$$

where k=n₁/n₂, h is the height of the cylindrical part of the micro-lens, α is the incident angle of the illumination light to the spherical surface of the micro-lens.

In the next step, a small drop of antibody solution is added into the sample wells of said micro-lens multi-well plate 102, said central control module 105 then immediately works with said intelligent image auto-recognition and data analysis software unit to control said intelligent auto-scanning and imaging module 104 to capture a series of images of said micro-lens, and deduces the information about the variation of the refractive index in the solution during Ag-Ab reaction process from the obtained images in the same way as mentioned in [0019].

By averaging the refractive indices of the Ag-Ab solution obtained from the 10^{th} to the 100^{th} second after adding the antibody solution, said central control module 105 works with said intelligent image auto-recognition and data analysis software unit to achieve an average refractive index n₁ of the solution after the startup of the Ag-Ab reaction.

said central control module 105 then works with the intelligent image auto-recognition and data analysis software unit to compare the averaged n₁ value with that before the Ag-Ab reaction n₁₀ to obtain a relative refractive index change Δn before and after the Ag-Ab reaction.

Since antigen-antibody reaction or ligand-receptor reaction can induce refractive index change Δn in sample solution, and Δn is a function of the amount of antigen-antibody complexes, so said central control module 105 can work with said intelligent image auto-recognition and data analysis software unit to determine whether there is antigen-antibody reaction and the amount of antigen or antibody in the sample solution according to the relation between Δn and the concentration of antigen which is obtained previously, and shows the results on said touchscreen displaying module 106.

The similar process from [0018] to [0023] can be completed to detect antibody in a sample solution by using the presently claimed immunoassay apparatus while replacing antibody solution mentioned in [0020] with antigen solution.

Said temperature controlled transparent toughened glass platform 103, said intelligent auto-scanning and imaging module 104, said central control module 105, and said touchscreen displaying module 106 are integrated into an apparatus main box 108, this enables said immunoassay apparatus to be a compact and portable apparatus for on-site immunoassay.

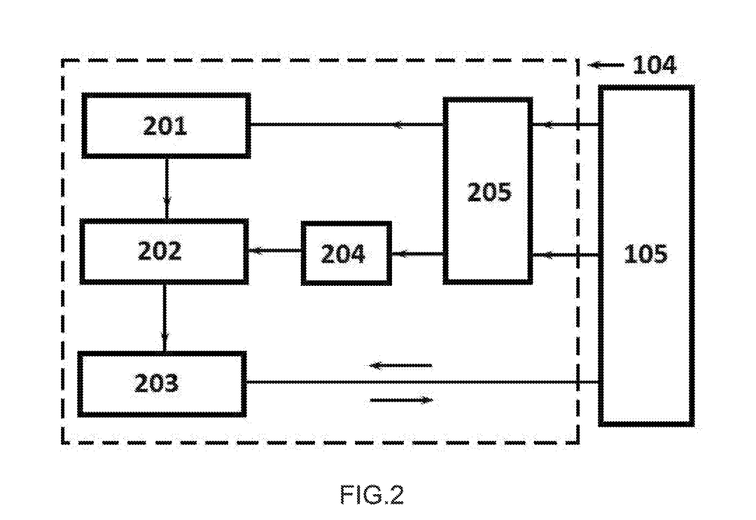

FIG. 2 is the structure of said intelligent auto-scanning and imaging module 104. Referring to FIGS. 1 and 2, central control module 105 controls galvanometer optical scanner 201 to scan each of the micro-lenses in micro-lens multi-well plate 102, and at the same time controls driving control circuit card 205 to drive driving motor 204 to adjust the focus of objective lens 202, and controls high resolution digital camera 203 to perform in focus imaging of the micro-lenses.

### DETAILED DESCRIPTION OF A PREFERRED EMBODIMENT

The following examples and drawings depict an implementation of the presently claimed invention in further detail.

In a first illustrative example:

The immunoassay apparatus is a 2-path compact device with size of 25 cm×20 cm×25 cm, it comprises a monochromatic light source module, an intelligent auto-scanning and imaging module, a temperature controlled transparent toughened glass platform, a central control module with specially designed software unit for intelligent micro-lens image auto-recognition and data analysis, and a touchscreen displaying module.

The monochromatic light source module of said immunoassay apparatus is a LED light source emitting 535 nm monochromatic parallel light with an angle of divergence less than 0.2° and a beam size of 2 cm in diameter.

The intelligent auto-scanning and imaging module comprises an one-axis galvanometer optical scanner, a high resolution digital camera with pixels of 14 M and frame rate of 13 fps, an autofocus 4× objective lens, a driving motor and a driving control circuit board to control said optical scanner and said driving motor for auto-scanning and autofocus imaging of said micro-lens. The module can autofocus on the micro-lens in said micro-lens test well plate and form distinct image of the micro-lens in <20 s.

The temperature controlled transparent toughened glass platform is a transparent toughened glass board with a thin film of IndiumTinOxide and controlled by a high accuracy XMT8008GP PID (proportional-integral-derivative) temperature controller. It enables the temperature of the sample in said micro-lens test well plate to rapidly reach to set temperature in 2 min and maintain with an accuracy of ±0.1° C.

The central control module is a Gigabyte BXi3-5010 microcomputer with size of about 15 cm×15 cm×5 cm. Its CPU is ≥2.1 G Hz, memory: 4G, hard disk: 200 G.

The touchscreen displaying module comprises a 10 inches capacitive touch screen and acting as the displaying screen of the immunoassay apparatus for displaying control touch keys and images of micro-lenses an resulting data.

The intelligent image auto-recognition and data analysis software unit is built based on Visual C⁺⁺6.0. It enables rapid intelligent image auto-recognition and data analysis on the image of micro-lenses to monitor the instantaneous variation of refractive index during the antigen-antibody reaction process in the solution, and determine the concentration of antigen/antibody in solution.

The present apparatus permits one to perform measurement on Ag-Ab reaction in solution with very low sample volume (several μL) and high accuracy, reliability, repeatability, and its detection limit is as low as several pg/mL, can finished measurement within two minutes while without any labeling, expensive enzymes, pre-immobilization/modification, and post-washing.

The present apparatus also permits one to monitor the dynamic process of Ag-Ab reaction in real time to provide kinetic and thermodynamic parameters such as association and dissociation rate constants about the reaction. It is able to perform detection on colored and severely hemolysis clinical samples as well.

In a second illustrative example:

The immunoassay apparatus is a compact 16-path apparatus which can perform 16-path Ag-Ab measurement simultaneously and with a size of 28 cm×23 cm×26 cm.

The monochromatic light source module of said immunoassay apparatus is a LED light source emitting 590 nm monochromatic parallel light with an angle of divergence less than 0.2° and a beam size of 5 cm in diameter.

The intelligent auto-scanning and imaging module comprises a X-Y 2-axis galvanometer optical scanner, a high resolution digital camera with pixels of 18 M and frame rate of 25 fps, an autofocus 2× objective lens, a driving motor and a driving control circuit board to control the optical scanner and driving motor for auto-scanning and autofocus imaging of micro-lens. The module can autofocus on the micro-lens in a micro-lens test well plate and form distinct image of the micro-lens in <10 s.

The central control module is a Gigabyte BKi5HA-7200 microcomputer with size of 12 cm×11 cm×4.6 cm. Its CPU is ≥2.5 G Hz, memory: 4G, hard disk: 800 G, and its screen is 12 inches capacitive touch screen.

The other parts of the present apparatus are the same as the first illustrative example.

Since certain changes may be made in the above apparatus without departing from the scope of the invention herein involved, it is intended that all matter contained in the above description or shown in the accompanying drawings shall be interpreted as illustrative and not in a limiting sense. The presently claimed invention may also be applied in a manner not covered by the above-mentioned cases. Other approaches may also be applied which do not deviate from the essence and spirit of the presently claimed invention. Foreseeable changes, modifications, substitutions, combinations or simplifications can be applied as equivalent methods and are included in the presently claimed invention within the scope of protection.

What is claimed is:

1. A compact multi-path micro-lens imaging micro-immunoassay apparatus based on the technique of micro-lens imaging for assaying fluid sample, comprising:
(a) a monochromatic light source module for illumination,
(b) an intelligent auto-scanning and imaging module for auto-scan imaging of micro-lenses immersed in antigen-antibody or other ligand-receptor solution,
(c) a temperature controlled transparent toughened glass platform for placing micro-lens multi-well test plate and controlling temperature,
(d) a central control module with specially designed software unit for controlling auto-scan imaging, intelligent micro-lens image auto-recognition and data analysis,
(e) a touchscreen displaying module for displaying operation touch keys and resulting images and data.

2. In an apparatus as defined in claim 1 wherein said monochromatic light source module is a monochromatic light source which emits monochromatic parallel light projecting to the spherical top of micro-lens with an angle of divergence less than 0.5° and a beam size greater than the size of micro-lens.

3. In an apparatus as defined in claim 1 wherein said intelligent auto-scanning and imaging module comprises a galvanometer optical scanner, a high resolution digital camera, an autofocus objective lens, a driving motor and a driving control circuit board to control said optical scanner and said driving motor for auto-scanning and autofocus imaging of said micro-lens.

4. In an apparatus as defined in claim 1 wherein said temperature controlled transparent toughened glass platform includes a transparent toughened glass board with a thin film of IndiumTinOxide, and a high accuracy PID temperature controller for placing micro-lens multi-well test plate and controlling the temperature of a test liquid sample which submerging said micro-lens to set temperature within 2 min and with accuracy ±0.1° C.

5. In an apparatus as defined in claim 1 wherein said central control module includes a microcomputer with specially designed software unit for controlling the operations of said apparatus including auto-scanning, autofocus imaging, intelligent image auto-recognition and data analysis.

6. In an apparatus as defined in claim 1 wherein said touchscreen displaying module is a touchscreen for displaying images of micro-lenses and resulting data, and with touch keys for controlling all the operations of said apparatus.

7. In an apparatus as defined in claim 3 wherein said galvanometer optical scanner is a high speed galvanometer optical scanner which is controlled by said control circuit board for alternate auto-scan imaging on different micro-lenses.

8. In an apparatus as defined in claim 3 wherein said autofocus objective lens is a 2×-10× objective lens driven by said driving motor which is controlled by said control circuit board for autofocusing.

9. In an apparatus as defined in claim 3 wherein said control circuit board comprises two motor motion controllers for respectively controlling the scanning of said galvanometer optical scanner and the motion of driving motor to drive said autofocus objective lens for focusing under the instructions of said central control module.

10. In an apparatus as defined in claim 3 wherein said high resolution digital camera is a digital camera with pixels ≥5 M, frame rate >10 fps for high resolution and rapid imaging of said micro-lens.

11. In an apparatus as defined in claim 1 wherein said central control module has specially designed software unit for auto-programmatic imaging on micro-lenses when distinct image is achieved.

12. In an apparatus as defined in claim 1 wherein said central control module has specially designed software unit to intelligently auto-recognize the image of micro-lens and measure the values of r and R in the image of micro-lens, and deduce the refractive index n₂ of Ag-Ab solution with equation (1) and the concentration of antigen or antibody in the solution according to the change of n₂ before and after the Ag-Ab reaction:
$$\frac{r}{R} = sin α - ( cos α + \frac{h - R}{R} ) \frac{sin α \sqrt{1 - {k}^{2} {sin}^{2} α} - k sin α cos α}{cos α \sqrt{1 - {k}^{2} {sin}^{2} α} + k {sin}^{2} α} , \qquad ( 1 )$$
where k=n₁ is the refractive index of micro-lens, h is the height of the cylindrical part of micro-lens, α is the incident angle of the illumination light to the spherical surface of micro-lens.

13. In an apparatus as defined in claim 1 wherein said touchscreen has data displaying windows and control touch keys for displaying resulting images and data, and all the operation of said apparatus.

14. In an apparatus as defined in claim 1 wherein said apparatus is an compact portable apparatus, all of its modules except said light source module are integrated into an apparatus main box with said temperature controlled transparent toughened glass platform as the top cover, to let said micro-lens multi-well plate or transparent petri dish containing micro-lenses be directly placed on said toughened glass board.

15. In an apparatus as defined in claim 1 wherein said monochromatic light source module is hung over said temperature controlled transparent toughened glass platform in said apparatus main box by a foldable support mounted on the rear surface of said apparatus main box.
